(19) United States
(12) Patent Application Publication
(10) Pub. No.: US 2003/0136415 A1
(43) Pub. Date: Jul. 24, 2003

(54) Non-invasive erectile dysfunction aid

(76) Inventor: Ralph H. Lanton JR., Ferguson, MO (US)

Correspondence Address: Gregory P. Gadson, Esq., 19375 Amber Way, Noblesville, IN, 46060, US

(21) Appl. No.: 10/054,178

(22) Filed: Jan. 19, 2002

(51) Int. Cl. A61F 6/02

(52) U.S. Cl. 128/842

(57) ABSTRACT

A simple, non-invasive artificial erection apparatus for erectile dysfunction sufferers has a semi-rigid tube with an inflatable bladder coupled to its inside wall. The apparatus also contains a valve and a pump for inflating the bladder through the valve. When inflated, the bladder forms a contracting lumen that provides pressure around the penile shaft to provide the artificial erection. The valve allows the bladder to substantially deflate the bladder for removal of the apparatus, or incrementally deflate to adjust the pressure of the bladder for comfort during intercourse. Other aspects of the present invention include, inter alia, peristaltic inflation of the bladder to help congregate blood around the penile end, the ability to adjust the length of the apparatus by unrolling (or re-rolling) the distal end of the apparatus, which integrates the tube and bladder.

### FIELD OF THE INVENTION

The present invention generally relates to non-pharmacological therapies for erectile dysfunction, which allow the artificial achievement or maintenance of erections.

### BACKGROUND OF RELATED ART

Erectile dysfunction can be broadly defined as the difficulty to, or the inability to, achieve or maintain a penile erection. While there is no age floor, erectile dysfunction is especially prevalent in older men who may still have an interest in sexual intercourse.

Prior art remedies—both short-term and long-term-have met with only limited success, and have serious drawbacks.

Simple remedies, such as placing tight-fitting rings at the base of the penis have been used to help congregate blood towards the penis tip, in the hope that the extra blood will cause an erection. However, not only are such rings uncomfortable, but they also do not produce erections in many erectile dysfunction sufferers.

Invasive therapies may offer better results, depending on how “better” is defined. One example of an invasive approach involves surgically implanting a semi-rigid stiffener in the body of the penis. The patient can then manipulate the penis to the correct attitude for all functions, such as urination (perhaps down or straight out), or artificial erection (perhaps up). Such an approach is often uncomfortable, and often feels extremely unnatural to the patient. It is also worth reiterating that such an approach requires surgery, and can therefore be expensive, and lead to complications from the surgery.

Another approach involves surgically implanting a balloon in the shaft of the penis with the balloon tube/stem exposed to the skin surface. When an erection is desired, an external pump that can be manually or machine-operated transcutaneously introduces air into the implanted balloon to stiffen the shaft of the penis. This approach is invasive, expensive to implement, may be uncomfortable to the patient, and risks infection to the body tissue around the tube area.

Still other prior art approaches are pharmacologically based. For example, drugs that purport to cause erections can be injected via a hypodermic needle into the penis. Other drugs such as Viagra® are orally consumed, and purport to increase blood flow to the penis to cause an erection. These approaches can be expensive, may cause unwanted physiological side effects, and are not effective for some men who, even when their member becomes engorged, may not also have the transverse rigidity needed for intercourse.

The above prior art approaches are illustrative, and not exhaustive. However, whether the approaches detailed supra are used, or others, all have been found to have major drawbacks. What is therefore greatly needed but unavailable in the prior art, is a simple, effective, non-invasive, and inexpensive approach to providing or maintaining erections when desired by an erectile dysfunction sufferer.

### SUMMARY OF THE INVENTION

In view of the above-identified problems and limitations of the prior art, the present invention provides an artificial erection apparatus for introducing or maintaining a penile erection. The apparatus at least includes a semi-rigid, support wall adapted to at least partially encircle a penis, an annular, inflatable bladder coupled to the inside of the support wall, a bladder valve coupled to the bladder, the valve adapted to control the ingress and egress of a bladder-inflating fluid, and a pump coupled to the bladder valve, the pump adapted to introduce the bladder-inflating fluid into the bladder. The bladder is adapted to form a lumen through which a penis is inserted, wherein the expansion of the bladder introduces a distributed force around the penis shaft surface to artificially create or maintain an erection. The length of the support wall is adaptable to at least expose the glans during an artificial erection.

The present invention also provides an artificial erection method for introducing or maintaining a penile erection. The method at least includes the steps of providing a semi-rigid, support wall adapted to at least partially encircle a penis, coupling an annular, inflatable bladder to the inside of the support wall, and via a bladder valve coupled to the bladder, controlling the ingress and egress of a bladder-inflating fluid. The method also at least includes the steps of, via a pump coupled to the bladder valve, introducing the bladder-inflating fluid into the bladder, via the bladder, forming a lumen through which a penis is inserted, and expanding the bladder to introduce a distributed force around the penis shaft surface to artificially create or maintain an erection. The length of the support wall is adaptable to at least expose the glans during an artificial erection.

The present invention is described in detail below, with reference to the drawings.

### BRIEF DESCRIPTION OF THE DRAWINGS

Features and advantages of the present invention will become apparent to those skilled in the art from the following description with reference to the drawings, in which:

FIG. 1 shows a top view and a side, cutaway view of the preferred embodiment of the present-inventive artificial erection apparatus with the bladder in an uninflated state;

FIG. 2 shows a top view and a side, cutaway view of the preferred embodiment of the present-inventive artificial erection apparatus with the bladder in an inflated state;

FIG. 3 shows a side view of an alternate embodiment of the present-inventive artificial erection apparatus having a wrap-around support wall, top views of the bladder in the uninflated and inflated states, and a view of an unwrapped support wall;

FIG. 4 is a side view of another alternate embodiment of the present-inventive artificial erection apparatus with an adjustable length;

FIG. 5 shows a side view of a further alternate embodiment of the present-inventive artificial erection apparatus having a c-shaped columnar support wall, and top views of the bladder in the uninflated and inflated states;

FIG. 6 shows an isometric illustration of yet another alternate embodiment of the present-inventive artificial erection apparatus; and

FIG. 7 shows the embodiment of FIG. 6 with a reduced length for a user who has achieved a partial erection on his own.

### DETAILED DESCRIPTION OF THE PREFERRED EMBODIMENTS

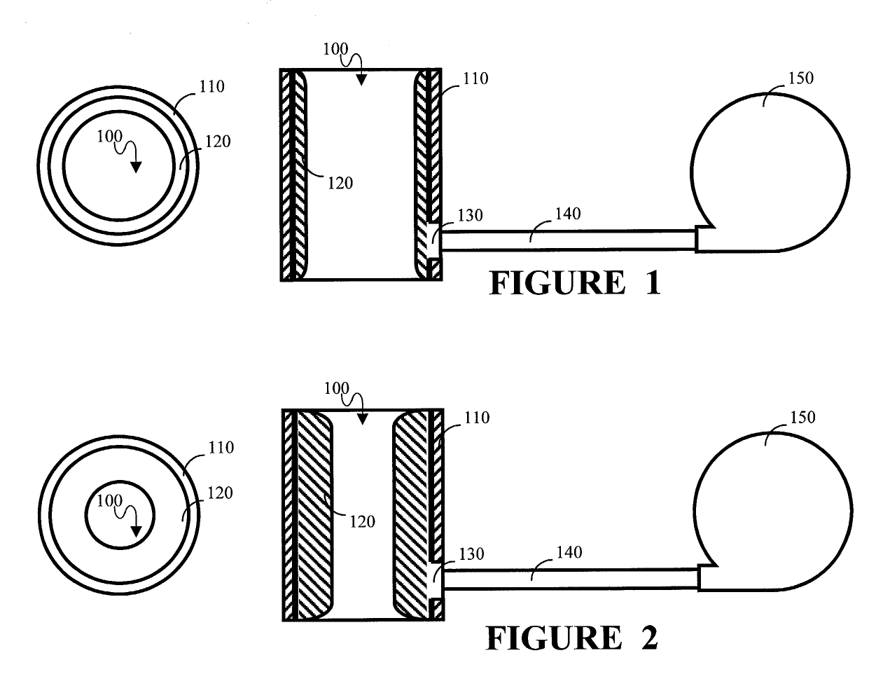

The top view and cutaway side view of FIG. 1 illustrate the basic embodiment of the present-inventive artificial erection apparatus 100 capable of causing and/or maintaining a penile erection. The main components of the apparatus are: a support wall 110 providing the rigidity needed to resists the transverse movement (during intercourse) of a penis inserted in the apparatus; a bladder 120 affixed to the inside of the support wall, which bladder expands to apply pressure directly to the shaft of the penis; a pump or aspirator 150/140 which serves to allow an erectile dysfunction patient (or user) to inflate the bladder; and a valve 130 which allows air from the pump to inflate the bladder, but closes to maintain bladder pressure after inflation.

To operate the artificial erection apparatus 100, the user places the penis through the lumen of the apparatus while the bladder 120 is in an uninflated, or no more than partially inflated state. The top view in FIG. 1 shows the bladder 120 in the uninflated stated to allow easy insertion. The pump tube 140 is inserted into the valve 130, followed by the pumping of air into the bladder to uniformly or nearly uniformly place pressure around the shaft of the penis. The top view and cutaway side view of FIG. 2 illustrate the apparatus 100 with the bladder 120 inflated and expanded to engage with the penis to support an artificial erection. In the preferred embodiment, the bladder is designed to inflate in a peristaltic manner with the initial inflation beginning at the base of the apparatus, and then moving outward to the distal end of the apparatus to produce a greater concentration of blood toward the penis tip. The peristaltic inflation feature, however, need not be included to make the present-inventive apparatus function generally.

In the preferred embodiment, the support wall 110 is in the form of a semi-rigid tube having a fixed diameter and length. The length of the support wall is a matter of design choice, but would not extend beyond the glans when the apparatus is rested against base of the penis. The stiffness of the support wall can be inherent in the material, or if the stiffness of the chosen material is not sufficient, stiffeners can be embedded in the support wall either during the manufacturing process, or by allowing the user to retrofit the apparatus with stiffeners.

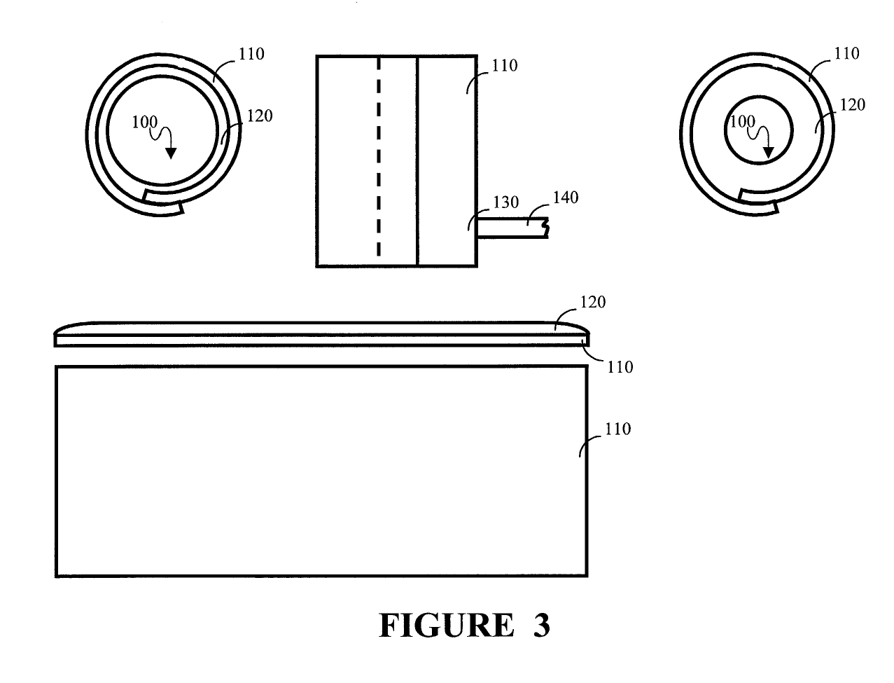

However, the support wall 110 need not be a manufactured tube as shown in FIGS. 1 and 2, but can be constructed in other ways. For example, the support wall 110 can be in the form of a flexible sheet with the bladder attached on one side, as shown in FIG. 3. In this case the “tube” is formed by overlapping the ends and attaching them to create a custom diameter for the user. Once wrapped around, the ends of the sheet can be held together by a variety of means including an adhesive on the surface that allows easy separation when the surfaces are moved away from each other in an orthogonal direction, but the surfaces adhere when forces (such as tension) in the plane of the meshed surfaces are applied. Alternatively, the ends can be attached with various fasteners such as peg and hole combinations.

Another embodiment of an apparatus with an adjustable diameter is shown in FIG. 5. The support wall 110 is a pre-formed, c-shaped column with the ability to be opened when placed around a penile shaft to create custom diameters.

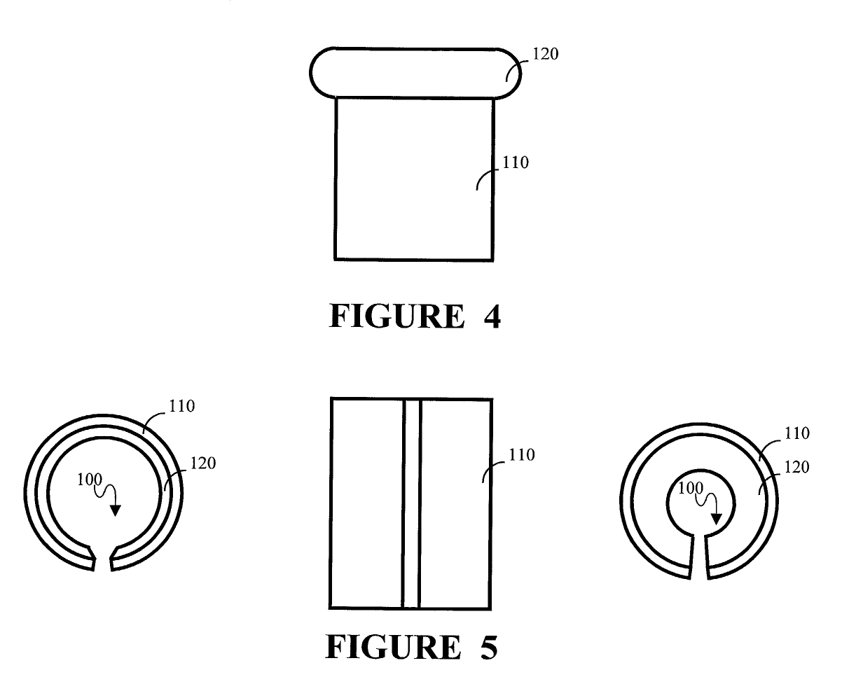

While the length of the support wall is fixed in the preferred embodiment, it need not be so to practice the novel aspects of the present-inventive artificial erection apparatus 100. While the support wall is semi-rigid, it can be flexible enough to allow it and the bladder to be rolled out to increase the length of the apparatus for a custom fit. The length can also be decreased by rolling the support wall and bladder in the opposite direction. This embodiment is illustrated in FIG. 4, and has similarities of function to unrolling a typical packaged condom.

The pump 150 can be as simple as an aspirator bulb, or it can be more complex, while serving the same function.

The valve 130 can be in the form of a simple, flexible check valve which is opened to allow air flow into the bladder when the pump tube 140 is wedged therein. The user can reduce the bladder pressure by squeezing the valve to allow air to escape incrementally for either a more comfortable fit, or for deflation of the bladder when artificial erection is no longer needed. The valve may of course be more complex and even contain moving parts such as a rotatable switch.

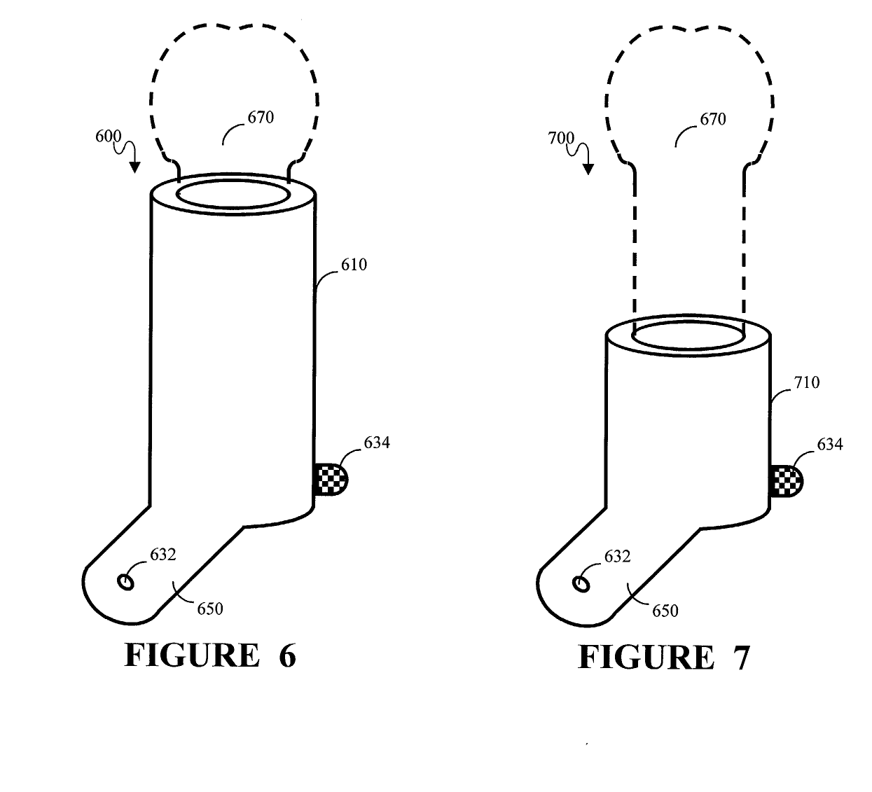

FIGS. 6 and 7 show yet another alternate embodiment of the present-inventive artificial erection apparatus. The apparatus 600 in FIG. 6 has a length (i.e., the measure of the support wall 610) appropriate for a user who has not achieved any noticeable degree of an erection. The apparatus 700 in FIG. 7 is identical to the one 600, except that it is shorter to accommodate a user who has achieved a partial erection, since it is anticipated that less transverse support is needed. The shorter length of the apparatus 700 allows for more exposure of the male organ 670 during intercourse.

The apparatuses 600 and 700 are distinguished from the apparatus 100 in that they integrate the pump 650 into the same member incorporating the support wall 610. Another distinguishing feature is the separation of the valve 130 into and air intake valve 632, an air release valve 634, and perhaps a check valve (not shown) directly connected to the bladder. In operation, the user covers the air intake valve while squeezing the pump 650 to force air through the check valve, thereby inflating the bladder. Between squeezes, the air intake valve is uncovered to allow the pump to refill with air. The release valve 634, responsible for deflating the bladder when activated, can have a variety of configurations, including those which squeeze, twist, screw (or unscrew), and the like.

Those skilled in the art to which the present invention pertains will appreciate that given the above description, the components of the present-inventive artificial erection apparatus can be manufactured from a variety of materials, including, inter alia, plastics and latex, as a matter of design choice.

Variations and modifications of the present invention are possible, given the above description. However, all variations and modifications which are obvious to those skilled in the art to which the present invention pertains are considered to be within the scope of the protection granted by this Letters Patent.

For example, the support wall need not form a traditional column. In an alternate embodiment, the proximal end of the support wall can have a skirt-like component to rest against the pubic area of the user and provide extra support of an artificial erection.

As another example, the valve and pump can be integrated into a common unit partially enclosed by the support wall, and slight exposed at the proximal end of the apparatus. Given the above description, those skilled in the art will be able to easily implement such modifications.

What is claimed is:

1. An artificial erection apparatus for introducing or maintaining a penile erection, said apparatus comprising:
a semi-rigid, support wall adapted to at least partially encircle a penis;
an annular, inflatable bladder coupled to the inside of said support wall;
a bladder valve coupled to said bladder, said valve adapted to control the ingress and egress of a bladder-inflating fluid; and
a pump coupled to said bladder valve, said pump adapted to introduce said bladder-inflating fluid into said bladder;
wherein said bladder is adapted to form a lumen through which a penis is inserted, wherein the expansion of said bladder introduces a distributed force around the penis shaft surface to artificially create or maintain an erection, and wherein the length of said support wall is adaptable to at least expose the glans during an artificial erection.

2. The apparatus in claim 1, wherein said bladder-inflating fluid is air.

3. The apparatus in claim 1, wherein the expansion of said bladder is peristaltic, starting at the proximal end of the penis, and moving toward the distal end of the penis.

4. The apparatus in claim 1, wherein said valve is adapted to, upon the action of a user, partially or fully deflate said bladder.

5. The apparatus in claim 1, wherein said support wall is a tube.

6. The apparatus in claim 1, wherein said bladder valve is comprised of a separate air intake valve and an air release valve.

7. The apparatus in claim 1, wherein said bladder and pump are part of a common unit that protrudes from the proximal end of said support wall.

8. The apparatus in claim 1, wherein said support wall comprises a sheet adapted to be fastened to itself and form a tube.

9. The apparatus in claim 1, wherein the distal ends of said support wall and said bladder are integrated to provide an adjustable apparatus length, wherein “unrolling” said apparatus lengthens it.

10. The apparatus in claim 1, wherein said support wall is a c-shaped column.

11. An artificial erection method for introducing or maintaining a penile erection, said method comprising the steps of:
providing a semi-rigid, support wall adapted to at least partially encircle a penis;
coupling an annular, inflatable bladder to the inside of said support wall;
via a bladder valve coupled to said bladder, controlling the ingress and egress of a bladder-inflating fluid;
via a pump coupled to said bladder valve, introducing said bladder-inflating fluid into said bladder;
via said bladder, forming a lumen through which a penis is inserted; and
expanding said bladder to introduce a distributed force around the penis shaft surface to artificially create or maintain an erection;
wherein the length of said support wall is adaptable to at least expose the glans during an artificial erection.

12. The method in claim 11, wherein said bladder-inflating fluid is air.

13. The method in claim 11, wherein the expansion of said bladder is peristaltic, starting at the proximal end of the penis, and moving toward the distal end of the penis.

14. The method in claim 11, further comprising the step of, via said valve, and upon the action of a user, partially or fully deflating said bladder.

15. The method in claim 11, wherein said support wall is a tube.

16. The method in claim 11, wherein said bladder valve is comprised of a separate air intake valve and an air release valve.

17. The method in claim 11, wherein said bladder and pump are part of a common unit that protrudes from the proximal end of said support wall.

18. The method in claim 11, wherein said support wall comprises a sheet adapted to be fastened to itself and form a tube.

19. The method in claim 11, wherein the distal ends of said support wall and said bladder are integrated to provide an adjustable apparatus length, further comprising the steps of “unrolling” said apparatus to lengthen it.

20. The method in claim 11, wherein said support wall is a c-shaped column.
